(19) United States
(12) Patent Application Publication
(10) Pub. No.: US 2019/0267133 A1
(43) Pub. Date: Aug. 29, 2019

(54) PRIVACY-PRESERVING METHOD AND SYSTEM FOR MEDICAL APPOINTMENT SCHEDULING USING EMBEDDINGS AND MULTI-MODAL DATA

(71) Applicant: NEC Laboratories Europe GmbH, Heidelberg (DE)

(72) Inventors: Maja Schwarz, Heidelberg (DE); Brandon Malone, Dossenheim (DE); Juergen Quittek, Leimen (DE); Mathias Niepert, Heidelberg (DE)

(21) Appl. No.: 15/976,894

(22) Filed: May 11, 2018

Related U.S. Application Data
(60) Provisional application No. 62/635,619, filed on Feb. 27, 2018.

(51) Int. Cl. G16H 40/20; G06Q 10/10

(52) U.S. Cl. CPC G16H 40/20; G06Q 10/1095

(57) ABSTRACT

An appointment scheduling device for scheduling an appointment for a patient to visit a health provider includes an embedder, a predictor, and a scheduler. The embedder receives input data about the patient. The input data is associated with a request to schedule the appointment with the health provider. The embedder generates an embedding based on the input data. The predictor receives the embedding and predicts an appointment parameter based on the embedding. The scheduler schedules the appointment based on the appointment parameter.

### CROSS-REFERENCE TO RELATED APPLICATION

Priority is claimed to U.S. Provisional Patent Application No. 62/635,619, filed on Feb. 27, 2018, the entire disclosure of which is hereby incorporated by reference herein.

### FIELD

The present invention relates to a method and system for medical appointment scheduling using embeddings that preserves privacy.

### BACKGROUND

In order to get an appointment with a desired physician, patients need to use a common channel to book the appointment. Traditionally, this has been done over the phone. Patients would call the practice/hospital and request a time slot that fits well with their schedule. Prior to getting the appointment, they would need to explain to the medical assistant what the reasons for the visit are. After the short conversation, the medical assistant would have enough information for triage. Based on this estimation of the appointment urgency, the current doctor's schedule, estimation of the time needed for the specific health issue and time availability of the patient, the medical assistant and the patient would agree on the suitable time slot. However, the medical assistants are using simple rules to estimate the appointment duration, which leads to poor accuracy and inefficient scheduling.

Currently, there are an increasing number of health providers that use computer systems to schedule physician appointments. For example, some providers allow patients to book appointments online (e.g., using a smart phone). However, these systems also use simple rules to estimate appointment duration and schedule appointments; and thus, suffer from similar problems of inaccuracy and inefficiency.

### SUMMARY

Embodiments of the present invention provide an appointment scheduling device for scheduling an appointment for a patient to visit a health provider. The appointment scheduling device includes an embedder, a predictor, and a scheduler. The embedder is configured to receive input data from the patient, the input data being associated with a request to schedule the appointment with the health provider, and configured to generate an embedding based on the input data. The predictor is configured to receive the embedding and to predict an appointment parameter based on the embedding. The scheduler is configured to schedule the appointment based on the appointment parameter.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 is a schematic system overview of system components of a system for medical appointment scheduling according to an embodiment of the present invention;

FIG. 2 is a simplified representation of a knowledge graph according to an embodiment;

FIG. 3 is a representation of embeddings according to an embodiment;

FIG. 4 is a schematic system and flow diagram illustrating the system and method for medical appointment scheduling according to an embodiment of the present invention;

FIG. 5 is a schematic system and flow diagram illustrating an embedding operation according to an embodiment of the present invention; and

FIG. 6 is a block diagram of a processing system according to an embodiment of the present invention.

### DETAILED DESCRIPTION

Large waiting times at hospital outpatient clinics are a cause of dissatisfaction to patients, cause additional stress to hospital staff, increase the risk of contagion, and add complications for patients with medical conditions. A recent report found that, in the U.S., the average wait time is 24 minutes, and that satisfaction declines as the waiting time increases, with 93.1% of patients being satisfied when waiting time is under five minutes, but only 84.9% satisfied when waiting time is over ten minutes (see Press Ganey, “Keep me waiting: Medical practice wait times and patient satisfaction,” Tech. Rep. (2009)).

While healthcare providers are increasingly relying on computer-based systems to schedule medical appointments, these systems are not sufficiently effective at reducing wait time; moreover, these systems do not efficiently allocate healthcare resources, and do not adequately maintain patient privacy. Moreover, traditional systems have no means to predict which patients might not show or come late. All these factors lead to poor appointment scheduling performance and result in high patient wait times and low satisfaction.

Currently, there are an increasing number of health providers that offer the option to book appointments online and use computer databases to track patent history. The inventors have recognized that this allows for an easy access to digitized data that could be used for various predictions (e.g., using machine learning models). Accordingly, embodiments of the present invention use the data provided by patients (patient input) at the time of making the appointment, as well as historic data if available, in order to make predictions impacting the scheduling decisions. These predictions can be used by a scheduler mechanism in order to reduce the time patients spend in the waiting room and increase the number of patients seen by doctors. In particular, in order to achieve this, the scheduler mechanism is provided with predictions on the appointment duration, no-shows and arrival punctuality.

Moreover, the inventors have recognized (and solved with the embodiments of the present invention) a technical challenge (based in the computer arts) regarding how to implement a scheduler that can use sensitive, private patient input (such as, description of the reasons for the visit in a natural language, and private images or videos), historic data, and patient profile data to predict the above mentioned factors and incorporate them into the scheduler. As such, the present invention provides an improved computer-based healthcare scheduling system, that uses multi-modal data and machine learning to accurately predict appointment duration, no-shows, arrival punctuality, and efficiently use healthcare resources, while preserving the privacy of the patient.

For example, embodiments of the present invention provide a method and a system for patient appointment scheduling based on machine learning models of relevant scheduling parameters. The method uses patient-provided data, such as a description of the health condition in natural language and relevant images, to accurately estimate the appointment duration. Using historic data, the method can also predict no-shows and late arrivals. Based on this information, the appointment scheduling is done so that the patient wait time is reduced and the number of patients seen by a given doctor is increased (compared to the current practice). Accordingly, the embodiments of the present invention directly effect improvements in the medical field, in particular medical appointment scheduling. Moreover, the method and system are privacy-preserving considering that only embeddings are stored the system (e.g., in a cloud server) in which the predictions are made.

Embodiments of the present invention are a substantial improvement over current systems because (for example) these current systems have patient appointment scheduling algorithms that do not use multi-modal input data, do not use machine learning models to make predictions (e.g., about appointment durations, no-shows and late arrivals), or do not adequately protect the privacy of patients. Instead, traditional computer-based scheduling systems use basic information (e.g., patient and doctor availability), are based on simple models (often hard coded or hand-manipulated) of appointment durations and other scheduling parameters, and generally use insecure methods of handling the patient's private data.

For example, U.S. Pat. No. 8,010,382 (the entire contents of which are hereby incorporated by reference herein) proposed a simple algorithm to schedule appointments that use patient estimations of appointment duration in the scheduling decisions. However, the inventors have found that patient estimation based systems are not accurate. U.S. Pat. No. 8,069,055 (the entire contents of which are hereby incorporated by reference herein) proposed applying machine learning models to predict the duration of a therapeutic procedure; however, in contrast to embodiments of the present invention, the proposed system does not use embeddings, does not contemplate using (and would nevertheless achieve significantly worse performance if it did) multi-modal data and missing data, and does not provide robust security to private information. Moreover, because the '055 Patent's model (and others) do not use all data modalities and/or account for missing data they lack certain aspects of the problem and information; and therefore, achieve lower performance.

Other simple patient-scheduling models have been proposed, including linear regression (see e.g., Strahl, Jonathan, “Patient appointment scheduling system: with supervised learning prediction,” Aalto University Master's Thesis (May 27, 2015), the entire contents of which are hereby incorporated by reference herein); heuristics (see e.g., Liu, Nan et. al, “Dynamic Scheduling of Outpatient Appointments under Patient No-shows and Cancelations,” Manu. Serv. Op. Management 12:2, 347-364 (2009), the entire contents of which are hereby incorporated by reference herein); and a scoring system (see e.g., U.S. Patent Pub. No. 2015/0269328, the entire contents of which is hereby incorporated by reference herein), but these simple methods, in contrast to embodiments of the present invention, are done without embeddings and without multi-modal data (descriptions in natural language, images, etc.), and produce unsatisfactory results (e.g., unsatisfying scheduling performance).

Indeed, embodiments of the present invention provide at least the following improvements to computer-based patient scheduling systems: (1) use of embeddings generated through patient appointment knowledge graphs; (2) prediction of scheduling relevant parameters based on multi-modal data (natural language descriptions of the health problem, photos, demographic data, etc.); (3) combining data modalities to allow the system to make predictions about patients for which no historical data exists; and (4) storing embeddings in servers (e.g., in cloud servers) to increase security for private information.

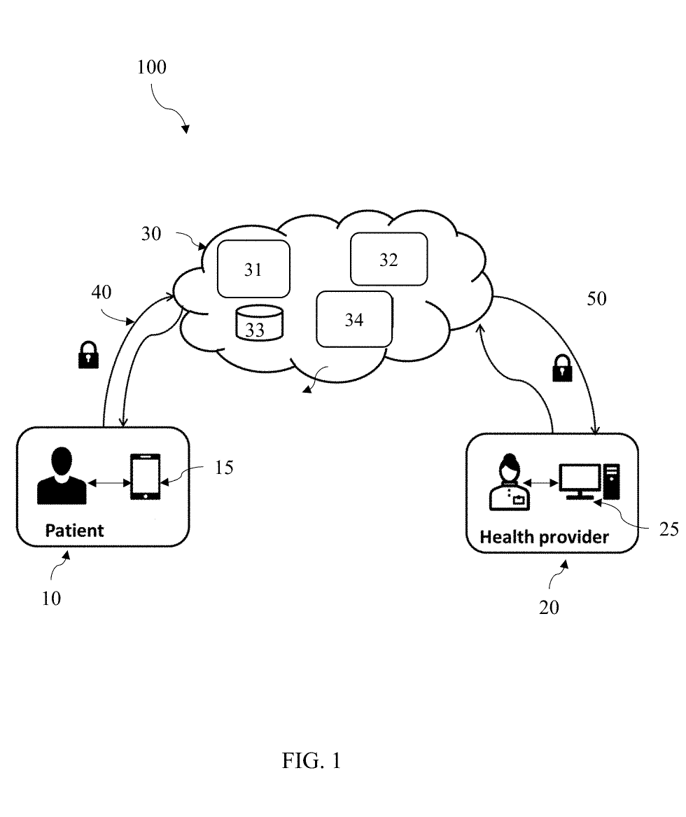

FIG. 1 is a schematic system overview of system components of a system for medical appointment scheduling according to an embodiment of the present invention.

FIG. 1 illustrates how a patient 10 and a health provider (e.g., a physician) 20 interact with the components of an appointment scheduling system 100.

The appointment scheduling system 100 collects relevant data from a patient 10 (patient input data) for use at an appointment scheduling device 30. Relevant data can include textual descriptions of reasons for a visit, related images, demographic data (such as age and gender), clinical data, past or chronic illnesses, allergies, etc.

The appointment scheduling device 30, may include, for example, a server (e.g., a cloud server), or at least one processor in communication with at least one memory (including a database). The appointment scheduling device 30 combines the patient input data with historical information (e.g., historical patient information), predicts the required time for an appointment, and schedules the appointment. The appointment scheduling system 100 can allow the health provider 20 to add outcomes (such as the duration of the appointment, arrival punctuality, no-shows, hints or suggestions for procedures to include in a physician's practice, and which medications the physician may consider providing) as historical information for use by the appointment scheduling device 30. Further, all communication occurs via secure channels 40, 50, which further assures privacy.

One particular technical improvement achieved by embodiments of the present invention is the appointment scheduling system's ability to predict the duration of the patient's appointment—as well as whether the patient will show up at all (likelihood of no-show)—even if that patient has never visited the heath provider before. In order for the appointment scheduling system to make such predictions, multiple modalities of data (for example, demographic information, textual descriptions, relevant images, etc.) characterizing the appointment are collected.

According to the embodiment of FIG. 1, the appointment scheduling system 100 can include the following components: (1) a communication device and interface 15 (e.g., a smart phone) used by the patient 10 to request an appointment; (2) an embedding model (e.g., an embedder) 31 that combines the patient input data (e.g., including patient's description of health problem or appointment needed) with historical data to create a representation of the appointment (i.e., the embedding); (3) a machine learning component (e.g., a predictor or prediction component) 32 that makes predictions (including, for example, predicting the likelihood of a no-show and/or the required time for the appointment) using the embedding as input; (4) a database 33 that stores historical data; (5) a scheduling system 34 that schedules the appointment based on the prediction; and (6) a communication and interface device 25 (e.g., a personal computer or local terminal) for the health provider 20.

According to an embodiment, the embedding model 31, the prediction component 32, the database 33, and the scheduling system 34 are all part of the appointment scheduling device 30. In some embodiments, each of these components may be deployed as part of a cloud computing system, which may be embodied by one or more (local or distributed) processors and (local or distributed) memories that are in communication with each other (e.g., over the internet).

The patient's communication and interface device 15 allows the patient 10 to request an appointment with the health provider 20. The communication and interface device 15 captures many modalities of input data, including patient demographics, textual descriptions of the purpose of the appointment (e.g., in natural language), and relevant images (e.g., picture of a portion of the patient's body that may require medical attention). For example, a patient 10 requesting an appointment due to a rash could take a picture of the rash with the communication and interface device 15 and then type in a natural language description of the symptoms. In some embodiments, a smartphone having an appropriate application installed and running is the communication and interface device 15. The communication and interface device 15 sends the patient input data to the embedding model 31 via an encrypted communication channel 40, which helps to preserve the patient's privacy.

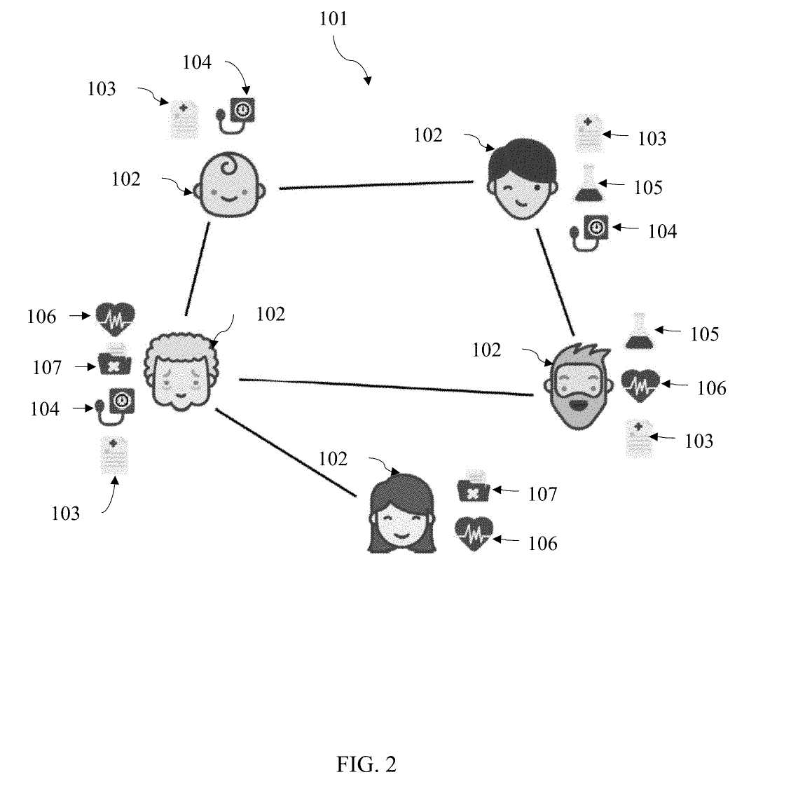

The embedding model 31 combines the modalities of patient input data (which can be structured in a knowledge graph of nodes connected based on their similarity) into a single, coherent representation of the appointment request. FIG. 2 illustrates a simplified patient knowledge graph according to an embodiment. Here the patient knowledge graph 101 illustrates a plurality of patients 102 and their respective multi-modal patient input data, including a textual description of the reason for the visit 103, a relevant picture associated with the reason for the visit 104, current medication information 105, vital statistics 106, and/or medical history 107. The relative distance and placement of the patients 102 on the knowledge graph, including their connections, indicate their similarity.

Referring back to FIG. 1, the embedding model 31 transforms each of the patient input data modalities into a dense vector representation. These dense vector representations for each of the patient input data modalities are then combined (e.g., concatenated) to create the embedding representation for the appointment.

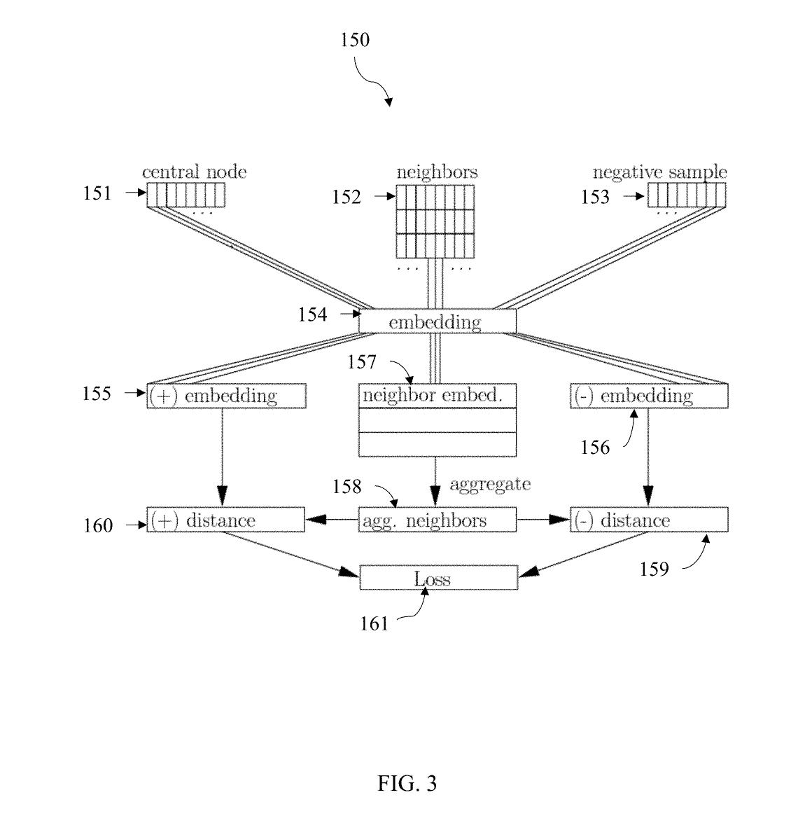

FIG. 3 illustrates an example embedding model according to an embodiment. Here, the embedding model 150, illustrates a central node 151, neighbors 152, a negative sample 153, an embedding 154, a (+) embedding 155, a (−)embedding 156, neighbor embeddings 157, (+) distance 158, (−) distance 159, an aggregate of neighbor embeddings 160, and loss 161. For a further discussion on embeddings, see Alberto Garcia-Durán and Mathias Niepert, “Learning Graph Representations with Embedding Propagation,” 31st Conference on Neural Information Processing Systems (2017) (“Garcia-Durán”), the entire contents of which are hereby incorporated by references herein.

In the embedding model 150 for a particular patient, the same input data modality for all neighbors of that patient are in the neighbor block 152. Also in the embedding model 150, the same input data modality for a random patient, −P, is included in the embedding graph. This random patient is commonly called a “negative sample” 153 in the academic literature. The embedding function 154 is a one-way “embedding function” that transforms input input data into the dense vector representation. This can be implemented as the “f_i” function from Garcia-Durán. The (+) embedding block is the dense vector representation of +P (i.e., the output of the embedding function 154 on the input data for +P). This can be implemented as “h_i(v)” from Garcia-Durán. The (−) embedding block 156 is the dense vector representation of −P (i.e., the output of the embedding function 154 on the input data for −P). This can be implemented as “h_i(u)” from Garcia-Durán. The neighbor embedding block 157 is the dense vector representations of each of +P's neighbors (i.e., the output of the embedding function 154 on the input data for each of neighbors 152. The application of an aggregation function (aggregate) which combines the dense representations of all neighbors into a single dense “summary” representation, for example, by taking the mean of each neighbor dense representations 157. This can be implemented as the “\tilde{g_i}” function from Garcia-Durán. The aggregate neighbors block 158 is the dense “summary” representation of all of +P's neighbors. This can be implemented as the “\tilde{h_i}(v)” from Garcia-Durán. The (+) distance 160 is the distance between +P's dense vector representation 155 and that of its aggregated neighbors 158. Any distance measure, such as Euclidean distance or (the inverse of) cosine similarity, is valid for this calculation. This can be implemented as “d_i” from Garcia-Durán. The (−) distance 159 is the distance between −P's dense vector representation 156 and that of +P's aggregate neighbors 158. The same distance measure used for (+) distance 158 should be used here, but there are no other constraints. Loss 171 is the difference between the (+) distance 160 and the (−) distance 159. This loss 161 is used for (re-)training the embedding model using standard optimization techniques. This can be implemented as Equation (3) from Garcia-Durán.

Contrary to other approaches, the embedding model 31 according to embodiments of the present invention handles missing data modalities by combining data from the new appointment request with similar ones in the appointment database. Similar appointment instances can be selected based on predefined similarity functions. For example, an embedding model 31 can ensure that the Euclidean distance between the dense vectors (including those of embeddings) of similar appointments is small. In an embodiment, a threshold is selected to determine what is small for 1/exp(d), where d is the Euclidean distance. The threshold is selected so that few edges are created, e.g., preferably less than 2% of the total maximal number, more preferably around 1% (e.g., between 0.75% and 1.25%). In other embodiments, a Manhattan distance or cosine similarity may be implemented as the predefined similarity function.

Also in contrast to other approaches, the transformation performed by the embedding model 31 is a one-way function. Accordingly, it is not possible to reconstruct the original patient information even if both the embeddings and the embedding model 31 are available, thereby providing more robust security and enhanced privacy to the patient information.

In the prediction component 32, a machine learning model uses the appointment representation from the embedding model 31 to make a prediction about the required time for the appointment, whether the patient will show up, as well as other relevant patient appointment parameters. The prediction model can be trained using historical outcomes from the appointment database.

The appointment duration prediction can be performed using a regression model (for example, interpretable models such as linear regression). The prediction whether the patient will show can be performed using a classification model (for example decision trees and logistic regression models). Furthermore, depending on the scheduling algorithm of a specific embodiment, models that classify the probability that the patient will come into multiple categories (e.g., more than the two categories which are present in show/no-show case) can be also used.

The appointment database 33 stores information on past appointments, including demographic, textual, and image data, as well as outcomes including whether the patient 10 was a no-show and the duration of the appointment. This includes historical data acquired before the appointment scheduling system 100 was set up, as well as data acquired through the appointment scheduling system 100, in order to increase the system's prediction performance. The initial content (anonymized historic data) can be used to train the machine learning components of both the embedding model 31 and the prediction component 32. Also, similar appointments can retrieved from the database when making new predictions or when data is missing (e.g., use historical embeddings and perform a Euclidian analysis to the present data to fill in missing data).

It is a particular security improvement according to an embodiment of the present invention that the privacy-sensitive data related to the appointment is not stored in the appointment scheduling system 100 (e.g., in the cloud or in the appointment database 33) in its original form, but in the form of embedding for privacy-preserving reasons. As described above, the transformation implemented by the embedding model 31 is a one-way function. Thus, even if the security of the database is compromised, the original patient information cannot be reconstructed.

Generated embeddings can be used to train the prediction component 32, in case there is feedback from the healthcare provider's 20 practice providing the actual appointment duration and information on no-shows. In order to retrain the embedding model 31, storing only embeddings is not sufficient. Hence, patients 10 that want to improve the system performance can opt-in and allow storing their data in anonymized form and using it to retrain the embedding model 31.

The scheduling system 34 is an algorithm deployed in the appointment scheduling device 30 (which may reside in the cloud). The scheduling system 34 assigns time slots to the requested appointments. The scheduling system 34 receives the predictions from the prediction component 32 and uses that information to schedule the patient's appointment in a manner that minimizes wasted time of the physician and patient waiting times. Additionally, the scheduling system 34 allows the health provider (e.g., physician) 20 to record the appointment results (e.g., whether the patient was a no-show and the duration of the appointment) in the appointment database 33. This recorded data can be used to retrain the prediction models of the prediction component 32. Communication from the scheduling system 34 is sent via a secure communication channel 40, 50.

The scheduling algorithm can include functionality for looking for available time slots of a predicted duration, scheduling patients with a higher no-show probabilities closer to the lunch-break or the end of the work day, and offering a couple of available time slots to a patient so that the patient can select the most suitable mode, among other functionalities.

The communication and interface device 25 for the health provider 20 allows, for example, doctors and medical assistants to access schedules and provide feedback that can be used to retrain the models (embedding and prediction models).

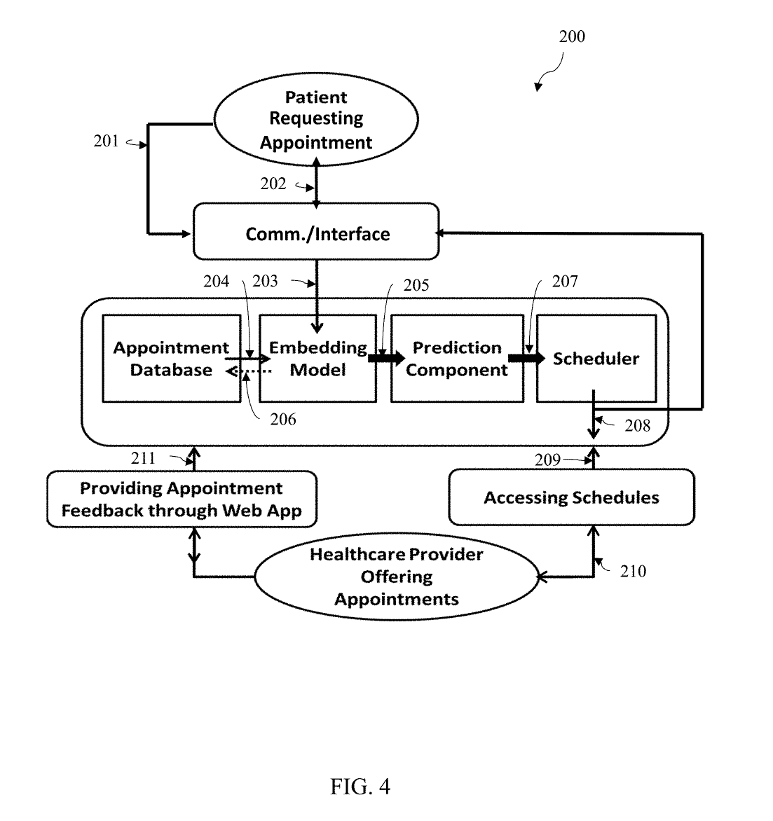

A flow chart illustrating the system and flow of a method according to an embodiment of the present invention is shown in FIG. 4.

According to an embodiment, a method for scheduling medical appointments 200 includes a patient (e.g., the patient 10) requesting an appointment (Operation 201). The patient may initiate this request via a communication and interface device (e.g., the communication and interface device 15), which can run an application for interfacing with an appointment scheduling device (e.g., the appointment scheduling device 30).

In response to initiating the request, patient input data is gathered and/or retrieved (Operation 202). In an embodiment, the patient input data is retrieved through an application running on the communication and interface device. The patient input data can be multi-modal data (e.g., various types of data including plaint language appointment request, schematized profile data, GPS data, images, etc.).

The communication and interface device then sends the patient input data (e.g., via the secure channel 40) to the appointment scheduling device (Operation 203). In an embodiment, the patient input data is sent to an embedder (e.g., the embedding model 31) of the appointment scheduling device. The embedder may retrieve historical patient data from an appointment database (e.g., the appointment database 33) (Operation 204); however, this is not required. The historical patient data may include historical data related to the patient that initiated the appointment request or other data (e.g., anonymized data or historical embeddings).

The embedder generates an embedding for the requested appointment, using the patient input data (e.g., the multi-modal input data)—and optionally the historical patient data—and sends the embedding to the predictor (e.g., the prediction component 32) (Operation 205). The embedder can also send the generated embedding to the appointment database for use in a later operation (Operation 206).

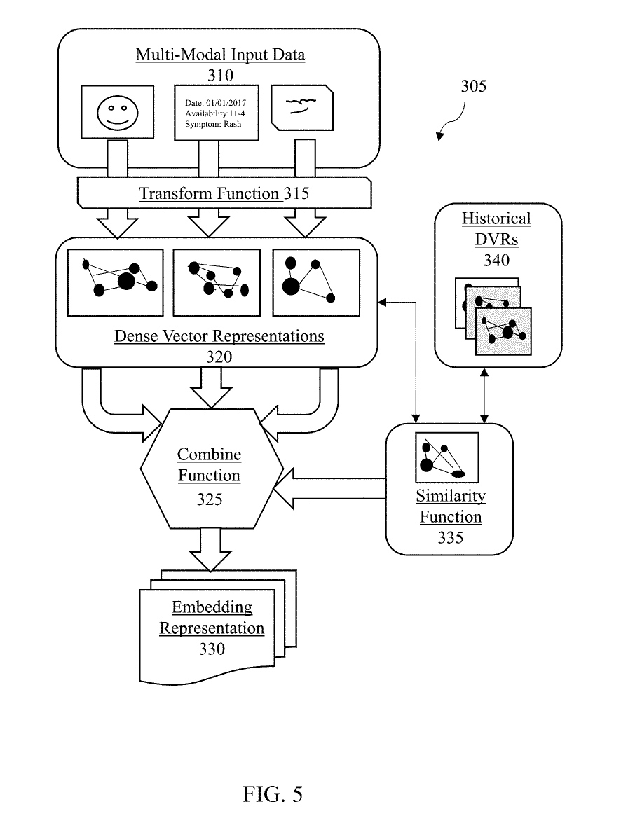

In an embodiment of the embedding operation illustrated in FIG. 5, the embedding operation combines the modalities of patient input data—which are structured in a knowledge graph of nodes connected based on their similarity—into a single, coherent representation of the appointment request. In particular, the embedding operation 305 transforms each of the patient input data modalities 310 into a dense vector representation 320 using a transform function 315. These dense vector representations 320 for each of the patient input data modalities are then combined to create the embedding representation for the appointment 330 using a combine function 325. In embodiments, dense vector representations 320, 340 are arrays of numeric values.

In the embedding operation according to embodiments, missing data modalities can be accounted for by combining data from the new appointment request with similar data from the appointment database. For example, as shown in FIG. 5, the embedding operation 305 can incorporate historical dense vector representation (DVRs) 340 into the embedding representation 330. Here, a similarity function 335 analyzes the DVRs 320 and the historical DVRs 340 to find similarities. The historical DVRs correspond to historical appointments, and may be stored in an appointment database (e.g., the appointment database 33). Similar historical DVRs 340 can be selected by the similarity function 325 based on a predefined similarity algorithm. For example, the similarity function 325 can find the most similar historical DVR 340 by determining which of the historical DVRs 340 has the smallest Euclidean distance as compared to the DVRs 320 of the current appointment request.

When using historical data to supplement the current request, the combine function combines one or more historical DVRs 340 selected by the similarity function 335 with the DVRs 320 to create the embedding representation 330.

Referring back to FIG. 4, the predictor predicts scheduling-relevant parameters (e.g., the required time for an appointment, whether the patient will show up, the starting time of an appointment, etc.) based on the received embedding, and sends the prediction to a scheduler (e.g., the scheduling system 34) (Operation 207).

The predictor provides the embedding (i.e., the embedding that represents the current appointment request) to one or more machine learning models to make one or more predictions about the scheduling-relevant parameters (e.g., predictions about the required time for the appointment, or whether the patient will show up, etc.). The predictor's machine learning models can be trained using historical outcomes from the appointment database.

In an embodiment, the predictor predicts the appointment duration using a regression model. In an embodiment, the predictor predicts whether the patient will show using a classification model. Furthermore, the predictor may use classification models that can classify the probability that the patient will come into multiple categories (e.g., more than the two categories which are present in show/no-show case: 50% likely to show; 75% likely to show; 80% likely to be 15 minutes late, etc.).

The scheduler makes an appointment schedule based on the received prediction, and sends the scheduled appointment to the patient's communication and interface device and the health provider's communication and interface device (e.g., the communication and interface device 25) (Operation 208).

In an embodiment, the scheduler may receive an appointment availability from the health provider (Operation 209) and additionally use the appointment availability when scheduling the appointment (Operation 208).

At any time, the healthcare provider offering appointments can access schedules and provide available appointment times to the appointment scheduling system, for example via its communication and interface device (Operation 210). The healthcare provider may also provide feedback on the appointment (e.g., actual appointment duration and whether or not the patient arrived or was late) to the appointment scheduling system (Operation 211), which can be used for training or for filling in missing data in subsequent embeddings.

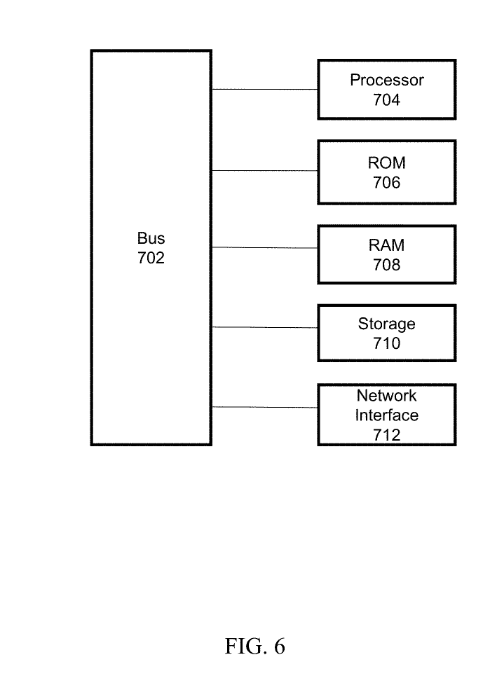

FIG. 6 is a block diagram of a processing system according to one embodiment. One or more processing system can be used to implement the protocols, devices, components, models, mechanism, systems and methods described above. The processing system includes a processor 704, such as a central processing unit (CPU) of the computing device or a dedicated special-purpose infotainment processor, executes computer executable instructions comprising embodiments of the system for performing the functions and methods described above. In embodiments, the computer executable instructions are locally stored and accessed from a non-transitory computer readable medium, such as storage 710, which may be a hard drive or flash drive. Read Only Memory (ROM) 706 includes computer executable instructions for initializing the processor 704, while the random-access memory (RAM) 708 is the main memory for loading and processing instructions executed by the processor 704. The network interface 712 may connect to a wired network or cellular network and to a local area network or wide area network, such as the Internet.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An appointment scheduling device for scheduling an appointment for a patient to visit a health provider, the appointment scheduling device comprising:
an embedder configured to receive input data about the patient, the input data associated with a request to schedule the appointment with the health provider, and to generate an embedding based on the input data;
a predictor configured to receive the embedding and to predict an appointment parameter based on the embedding; and
a scheduler configured to schedule the appointment based on the appointment parameter.

2. The appointment scheduling device according to claim 1, wherein the input data is multi-modal input data.

3. The appointment scheduling device according to claim 2, wherein the multi-modal input data comprises at least image data and natural language data.

4. The appointment scheduling device according to claim 1, wherein the embedder is configured to transform the input data into a knowledge graph of nodes connected based on their similarity.

5. The appointment scheduling device according to claim 4, wherein the knowledge graph of nodes connected based on their similarity is in a dense vector representation.

6. The appointment scheduling device according to claim 5, wherein the input data comprises at least first input data and second input data, and
wherein the embedder is configured to transform the first input data into a first dense vector representation, transform the second input data into a second dense vector representation, and combine the first dense vector representation and the second dense vector representation to generate the embedding.

7. The appointment scheduling device according to claim 6, the appointment scheduling device further comprising an appointment database configured to store a plurality of historical dense vector representations that individually corresponds to a particular historical patient appointment,
wherein the embedder is configured to identify a similar historical dense vector representation of the historical dense vector representations and combine the similar historical dense vector representation with the first dense vector representation and the second dense vector representation to generate the embedding.

8. The appointment scheduling device according to claim 6, wherein the embedder is configured to identify the similar historical dense vector representation based on determining which of the historical dense vector representations has the smallest Euclidean distance to one or both of the first dense vector representation and the second dense vector representation.

9. The appointment scheduling device according to claim 1, wherein the predictor is configured to use one or more machine learning models to predict the appointment parameter.

10. The appointment scheduling device according to claim 9, wherein the one or more machine leaning models comprises a regression model or a classification model.

11. The appointment scheduling device according to claim 1, wherein the appointment parameter comprises one or more of a required time for the appointment, whether the patient will show up, or timeliness of the patient's arrival.

12. The appointment scheduling device according to claim 11, wherein the predictor is configured to predict the required time for appointment using a regression machine learning model and to predict whether the patient will show up using a classification machine learning model.

13. A computer-implemented method of scheduling an appointment for a patient to visit a health provider, the method comprising:
receiving, by an embedder, input data about the patient, the input data associated with a request to schedule the appointment with the health provider;
generating, by the embedder, an embedding based on the input data;
receiving, by a predictor, the embedding;
predicting, by the predictor, an appointment parameter based on the embedding; and
scheduling, by a scheduler, the appointment based on the appointment parameter.

14. The computer-implemented method according to claim 13, wherein the input data is multi-modal input data.

15. A non-transitory computer readable medium comprising one or more instructions, which, when executed by a processor, cause the processor to perform the following operations:
receive input data about a patient, the input data associated with a request to schedule an appointment with a health provider;
generate an embedding based on the input data;
receive the embedding;
predict an appointment parameter based on the embedding; and
schedule the appointment based on the appointment parameter
